(19) United States
(12) Patent Application Publication
(10) Pub. No.: US 2011/0082467 A1
(43) Pub. Date: Apr. 7, 2011

(54) SURGICAL TOOL CALIBRATING DEVICE

(75) Inventors: Hung-Sheng Tien, Taoyuan (TW); Yen-Chu Chen, Taoyuan (TW); Jia-Jyun Hong, Taoyuan (TW); Chi-Bin Wu, Taoyuan (TW)

(73) Assignee: AccuMis Inc., Zhonghe City (TW)

(21) Appl. No.: 12/652,079

(22) Filed: Jan. 5, 2010

(30) Foreign Application Priority Data
Oct. 2, 2009 (TW) 098133468

(51) Int. Cl. A61B 19/00

(52) U.S. Cl. 606/130

(57) ABSTRACT

A surgical tool calibrating device for calibrating a surgical tool is provided. The surgical tool calibrating device includes a calibration block, an electronic sensing module, a diameter measurement module, and a signal communication module. The calibration block has a tool inserting portion in which the surgical tool can be inserted. The electronic sensing module is disposed on the bottom of the tool inserting portion for sensing the tip of the surgical tool and generating a measuring signal accordingly. The diameter measurement module is mounted around the tool inserting portion. The diameter measurement module is actuated to sense the surgical tool and measure the diameter of the surgical tool upon reception of the measuring signal. The signal communication module is electrically connected to the electronic sensing module and the diameter measurement module for transmitting the measuring signal.

### BACKGROUND OF THE INVENTION

1. Technical Field

The present invention relates to a surgical tool calibrating device and, more particularly, to a surgical tool calibrating device which is applicable to medical image scanning and configured for measuring the diameter of a surgical tool.

2. Description of Related Art

With continuous advancement in computer technology and the rapid development of three-dimensional medical imaging techniques, surgical operations that require high-precision positioning are now performed with the assistance of computers. Therefore, computer-assisted surgery is not only a major research field nowadays, but also an important trend in the future. For example, computers can be used as a surgical navigation tool in, among many others, brain neurosurgery, vertebral puncture, total knee replacement, and total hip joint replacement, and etc. However, all these surgical procedures demand extremely high precision and ample clinical experience because a slight error in the surgical path may injure the neighboring nerves. Hence, medical imaging and computer vision are combined in this computer-assisted surgical navigation system to help surgeons in performing high-precision surgical operations. Under such circumstances, it is of utmost importance to preoperatively ensure the dimension of surgical tools used in the computer-assisted surgical navigation system.

FIG. 1 illustrates schematically a surgical tool calibrating device 10 of a conventional surgical navigation system.

As shown in FIG. 1, the conventional surgical tool calibrating device 10 includes a calibration fixture 11, a calibration block 12, and an optical positioning device 13. The calibration fixture 11 is fixed in position to a first end 21 of a surgical tool 20 and includes a plurality of first position indicating elements 111. On the other hand, the calibration block 12 has a side provided with a plurality of second position indicating elements 121. The calibration block 12 further has another side which is adjacent to the aforesaid side and formed with a plurality of calibration bores 122 with different bore diameters. Thus, surgical tools 20 of various thicknesses can have their second ends 22 inserted into the calibration bores 122 with the corresponding bore diameters.

The optical positioning device 13 includes a detector 131 and a signal processor 132. The detector 131 is an infrared signal transceiver for transmitting an infrared signal (not shown). Meanwhile, the first and second position indicating elements 111 and 121 are provided with reflective markers for reflecting the infrared signal. The detector 131 receives the infrared signal reflected by the first and second position indicating elements 111 and 121 and thus obtains spatial coordinate information thereof. Since the first and second position indicating elements 111 and 121 are arranged with a fixed spatial relation therebetween, after receiving the spatial coordinate information transmitted from the detector 131, the signal processor 132 can determine the actual dimension of the surgical tool 20 by triangulation, and thereby calibrate the dimensional information of the surgical tool 20.

In practice, the surgical tool 20 is inserted by an operator into one of the calibration bores 122 with the appropriate bore diameter and is subsequently lowered to a specific depth. (The surgical tool 20 is usually inserted along the calibration bore 122 until the bottom of the calibration block 12 is reached.) Then, the operator manually activates a calibration procedure of the conventional surgical tool calibrating device 10. Therefore, if the operator inadvertently inserts the surgical tool 20 into a calibration bore 122 whose bore diameter does not match the surgical tool 20, or if none of the calibration bores 122 of the calibration block 12 has the matching bore diameter, calibration errors are bound to occur after the calibration procedure is activated. For obvious reasons, the operation described above tends to result in imprecise calibration and inconvenience of use.

### BRIEF SUMMARY OF THE INVENTION

It is an objective of the present invention to provide a surgical tool calibrating device, wherein an electronic sensing module and a diameter measurement module are integrated into a calibration block. As soon as the tip of a surgical tool makes contact with the electronic sensing module, the diameter measurement module is actuated to measure and thus obtain the correct diameter information of the surgical tool.

It is another objective of the present invention to provide a surgical tool calibrating device, wherein a diameter measurement module can be directly triggered to measure the diameter of a surgical tool, thereby enhancing the convenience in the use of the surgical tool calibrating device.

It is yet another objective of the present invention to provide a surgical tool calibrating device, wherein an electronic sensing module is configured not only for actuating a diameter measurement module to measure the diameter of a surgical tool, but also for triggering a calibration procedure. Thus, the precision of calibrating the diameter and length of a surgical tool can be effectively increased to prevent guiding errors which may otherwise result from inaccurate calibration.

In order to achieve the above and other objectives, the present invention provides a surgical tool calibrating device for calibrating a surgical tool, wherein the surgical tool calibrating device includes a calibration block, an electronic sensing module, a diameter measurement module, and a signal communication module. The calibration block has a tool inserting portion to be inserted by the surgical tool. The electronic sensing module is disposed on the bottom of the tool inserting portion and configured for sensing the surgical tool so as to generate a measuring signal accordingly. When receiving the measuring signal, the diameter measurement module, which is mounted around the tool inserting portion, is actuated to sense the surgical tool and measure the diameter thereof. The signal communication module is electrically connected to the electronic sensing module and the diameter measurement module and is configured for transmitting the measuring signal.

Implementation of the present invention at least involves the following inventive steps:

1. With the electronic sensing module disposed on the bottom of the calibration block, and due to the property of a piezoelectric element provided in the electronic sensing module, an electronic signal is generated as soon as the tip of a surgical tool contacts with the electronic sensing module. The electronic signal triggers the diameter measurement module mounted around the tool inserting portion so as to obtain information related to the diameter of the surgical tool.

2. After the diameter information of the surgical tool is precisely obtained, the diameter information is transmitted to an optical positioning device for reference, and a calibration procedure of the optical positioning device is simultaneously activated, thereby effectively enhancing the convenience and precision of surgical tool calibration.

### BRIEF DESCRIPTION OF THE SEVERAL VIEWS OF THE DRAWINGS

A detailed description of further features and advantages of the present invention is given below so that a person skilled in the art can understand and implement the technical contents of the present invention and readily comprehend the objectives and advantages thereof by reviewing the teachings disclosed herein and the appended claims in conjunction with the accompanying drawings, in which:

FIG. 1 is a perspective view of a surgical tool calibrating device of a conventional surgical navigation system;

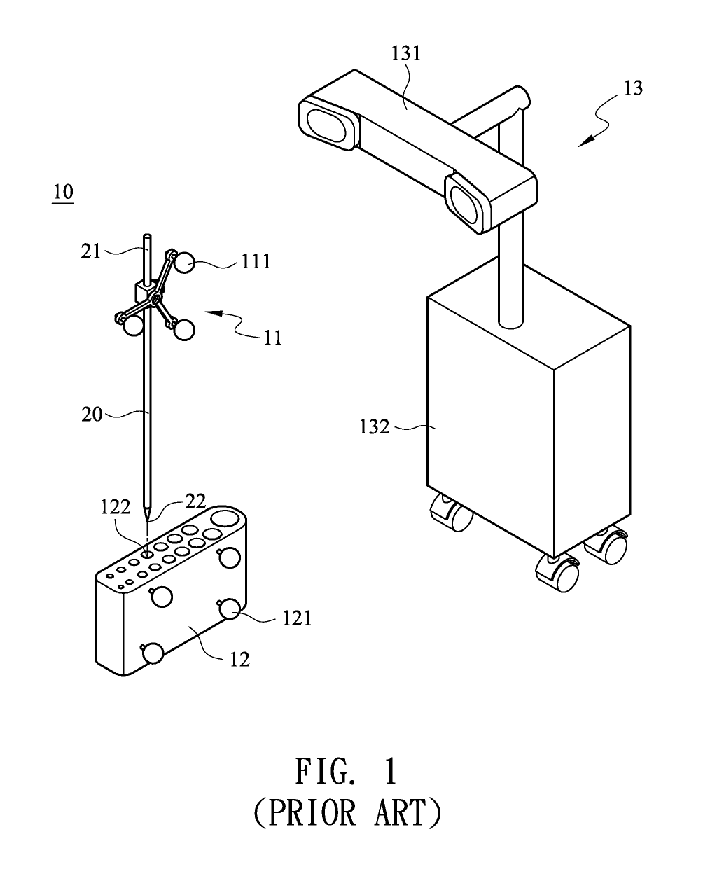

FIG. 2A is a perspective view of a surgical tool calibrating device according to the present invention, wherein a surgical tool has yet to be inserted into a tool inserting portion of a calibration block;

FIG. 2B is another perspective view of the surgical tool calibrating device according to the present invention, wherein the surgical tool has been inserted into the tool inserting portion; and

FIG. 3 is a function block diagram of the surgical tool calibrating device according to the present invention.

### DETAILED DESCRIPTION OF THE INVENTION

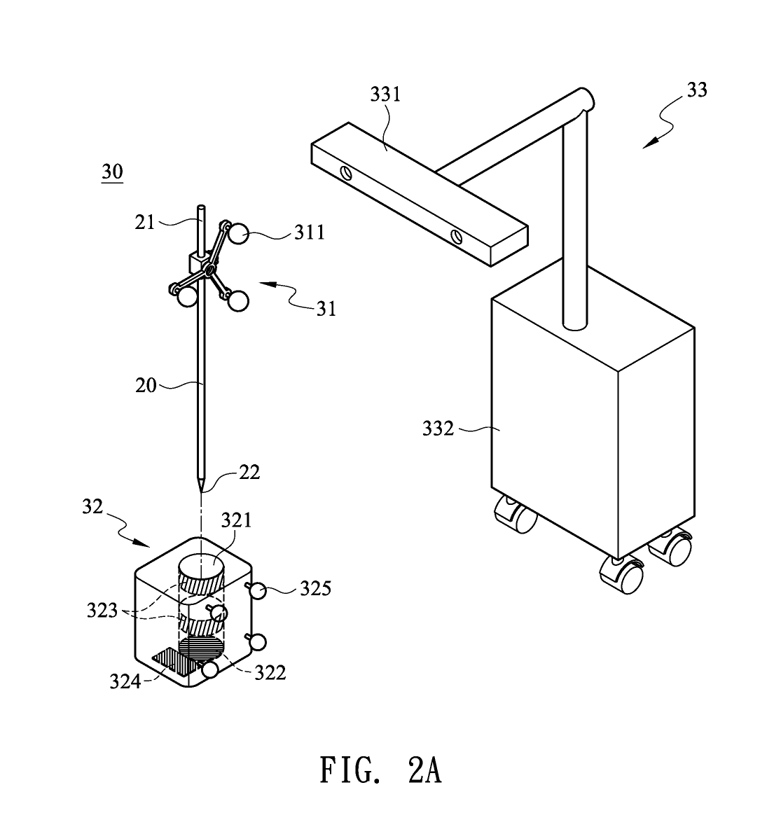

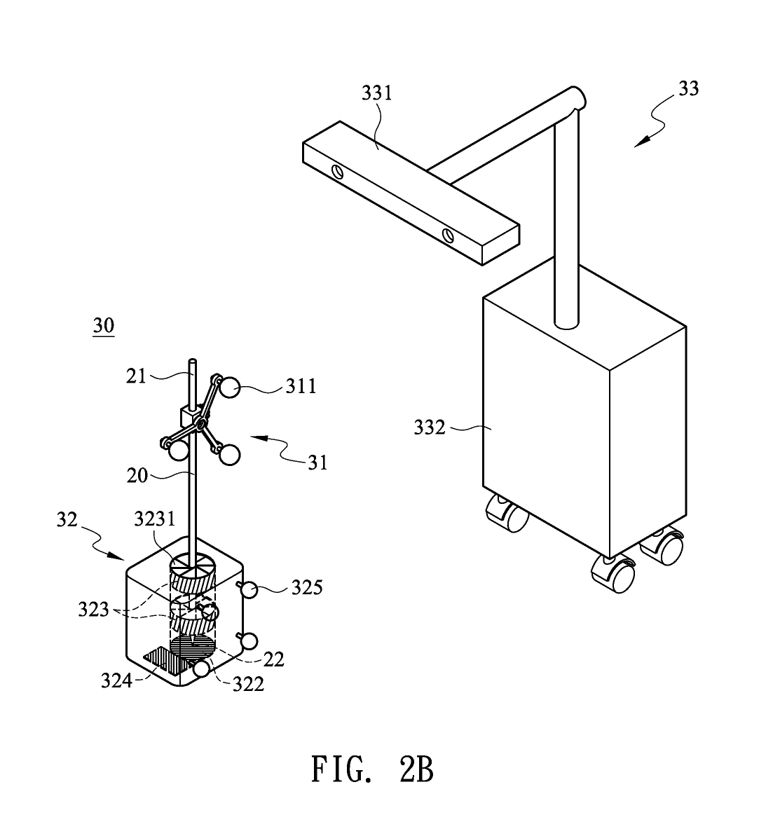

Referring to FIG. 2A and FIG. 2B for an embodiment of the present invention, a surgical tool calibrating device 30 for calibrating a surgical tool 20 essentially includes a calibration block 32, an electronic sensing module 322, a diameter measurement module 323, and a signal communication module 324. The calibration block 32 has a tool inserting portion 321 in which the surgical tool 20 can be inserted.

As in the prior art, a calibration fixture 31 is fixed in position to a first end 21 of the surgical tool 20 and includes a plurality of first position indicating elements 311.

Also as in the prior art, the calibration block 32 has a side provided with a plurality of second position indicating elements 325. The calibration block 32 further has a side which is adjacent and perpendicular to the aforesaid side and provided with the tool inserting portion 321 to be inserted by a second end 22 of the surgical tool 20. The tool inserting portion 321 is formed as a single calibration bore having a large bore diameter so as to accommodate surgical tools 20 of various sizes.

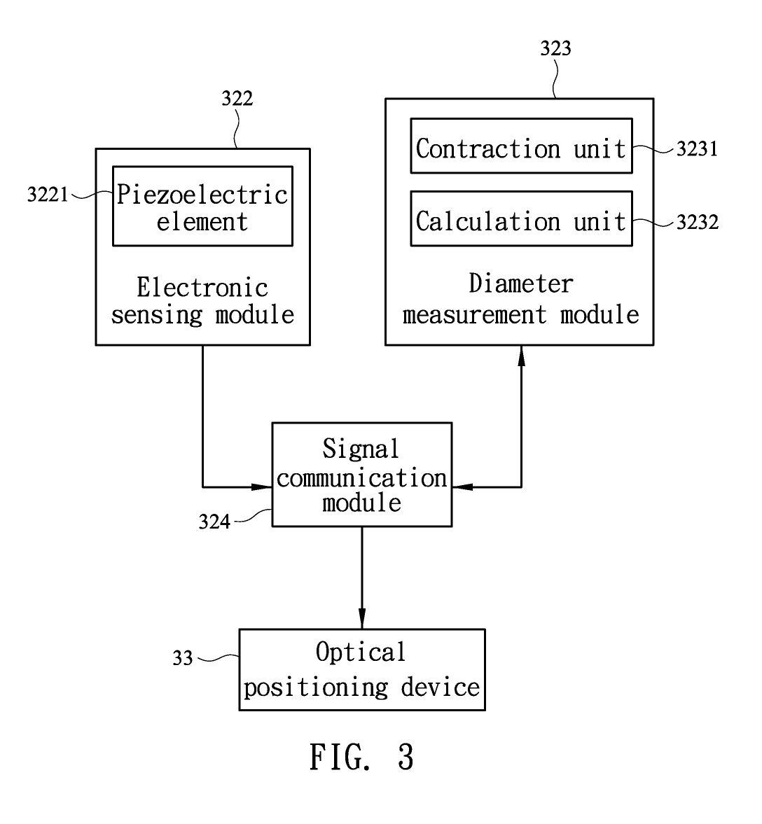

The electronic sensing module 322 is disposed on the bottom of the tool inserting portion 321 while the diameter measurement module 323 is mounted around the tool inserting portion 321. The signal communication module 324 is electrically connected to the electronic sensing module 322 and the diameter measurement module 323 (as shown in FIG. 3). When the surgical tool 20 is inserted in the tool inserting portion 321 and reaches a specific depth, the tip of the second end 22 of the surgical tool 20 makes contact with the electronic sensing module 322. Thereupon, the electronic sensing module 322 generates a measuring signal accordingly and transmits the measuring signal to the diameter measurement module 323 by way of the signal communication module 324. Thus, the diameter measurement module 323 is actuated to sense the surgical tool 20 and measure the diameter of the surgical tool 20.

With reference to FIG. 2B and FIG. 3, the diameter measurement module 323 further includes a contraction unit 3231 and a calculation unit 3232 (as shown in FIG. 3) electrically connected to the contraction unit 3231.

As shown in FIG. 2B, the contraction unit 3231 is a cylindrical structure composed of vanes. Moreover, the contraction unit 3231 is mounted in such a way as to surround the tool inserting portion 321. Therefore, when the diameter measurement module 323 receives the measuring signal generated by the electronic sensing module 322, the contraction unit 3231 contracts inward until it contacts with the surgical tool 20. As soon as the contraction unit 3231 makes contact with the surgical tool 20, the calculation unit 3232 calculates diameter information of the surgical tool 20 and sends the diameter information outward via an activating signal.

The signal communication module 324 further transmits the activating signal to an optical positioning device 33 and records the diameter information contained in the activating signal. Thus, calibration of the surgical tool 20 begins. The signal communication module 324 is a wire-based communication module or a wireless communication module; therefore, the signal communication module 324 transmits the measuring signal either wirelessly or via a wire.

The optical positioning device 33 includes a detector 331 and a signal processor 332, wherein the detector 331 is an infrared transceiver. In response to the activating signal transmitted by the signal communication module 324, the optical positioning device 33 starts an optical positioning process (e.g., triangulation) and carries out a calibration procedure for the surgical tool 20 in a way similar to the prior art, i.e., by sending an infrared signal and receiving a reflection thereof so as to determine the spatial coordinate information of each position indicating element. In order to reflect infrared signal emitted by the detector 331, the first and second position indicating elements 311 and 325 each include a reflective marker, reflective ball, or infrared reflector.

Please refer to FIG. 3 for a function block diagram of the present embodiment.

More specifically, since the electronic sensing module 322 includes a piezoelectric element 3221, a contact pressure is generated when the tip of the second end 22 of the surgical tool 20 makes contact with the electronic sensing module 322, as shown in FIG. 2B. Thus, an electronic signal is produced as the measuring signal and transmitted through the signal communication module 324 to the diameter measurement module 323, thereby causing the contraction unit 3231 of the diameter measurement module 323 to contract inward. When the contraction unit 3231 contracts inward and finally makes contact with the surgical tool 20, the calculation unit 3232 of the diameter measurement module 323 compares the area of the inwardly contracted vanes of the contraction unit 3231 with the original bore diameter of the tool inserting portion 321. The calculation unit 3232 then subtracts the area of the inwardly contracted vanes of the contraction unit 3231 from the original bore diameter of the tool inserting portion 321 to obtain information of the remaining space in the tool inserting portion 321, thus determining the diameter of the surgical tool 20.

The aforesaid diameter information is also in the form of an electronic signal and is transmitted to the optical positioning device 33 via the signal communication module 324. This electronic signal not only conveys the diameter information but also serves as the activating signal. Upon receiving the activating signal, the optical positioning device 33 activates a tool calibration procedure. Detection and calculation are then carried out, and the actual dimension of the surgical tool 20 is determined accordingly so as to calibrate dimensional information of the surgical tool 20.

As in the prior art, the first and second position indicating elements 311 and 325 are so arranged as to have a fixed spatial relation therebetween. Therefore, the only variables in the tool calibration procedure are the length of the surgical tool 20 (i.e., the distance between the first position indicating elements 311 and the tip of the second end 22 of the surgical tool 20) and the width of the surgical tool 20 (i.e., the diameter information calculated by the calculation unit 3232). By implementing of the present embodiment, an operator can make sure that the tip of the surgical tool 20 has been positioned at a specific position (i.e., the bottom of the calibration block 32) and that the length and width of the surgical tool 20 are determined. Hence, after the tool calibration procedure is triggered in real time, the precision of calibrating the surgical tool 20 can be significantly increased.

The foregoing embodiment is illustrative of the characteristics of the present invention so as to enable a person skilled in the art to gain insight into the contents disclosed herein and implement the present invention accordingly. The embodiment, however, is not intended to restrict the scope of the present invention. Hence, all equivalent modifications and variations which do not depart from the spirit and principle of the present invention should fall within the scope of the appended claims.

What is claimed is:

1. A surgical tool calibrating device for calibrating a surgical tool, the surgical tool calibrating device comprising:
a calibration block having a tool inserting portion in which the surgical tool can be inserted;
an electronic sensing module disposed on the bottom of the tool inserting portion and configured for sensing the surgical tool and generating a measuring signal accordingly;
a diameter measurement module mounted around the tool inserting portion and configured for sensing the surgical tool and measuring a diameter of the surgical tool upon receiving the measuring signal; and
a signal communication module, electrically connected to the electronic sensing module and the diameter measurement module, configured for transmitting the measuring signal.

2. The surgical tool calibrating device of claim 1, wherein the diameter measurement module comprises a contraction unit and a calculation unit.

3. The surgical tool calibrating device of claim 2, wherein the contraction unit is a cylindrical structure composed of vanes and surrounds the tool inserting portion such that, upon receiving the measuring signal, the contraction unit contracts inward so as to make contact with the surgical tool.

4. The surgical tool calibrating device of claim 3, wherein when the contraction unit makes contact with the surgical tool, the calculation unit calculates diameter information of the surgical tool and sends the diameter information via an activating signal.

5. The surgical tool calibrating device of claim 4, wherein the activating signal is transmitted to an optical positioning device through the signal communication module so as to begin calibration of the surgical tool.

6. The surgical tool calibrating device of claim 1, wherein the calibration block comprises a plurality of position indicating elements.

7. The surgical tool calibrating device of claim 6, wherein each said position indicating element comprises a reflective marker.

8. The surgical tool calibrating device of claim 6, wherein each said position indicating element is a reflective ball.

9. The surgical tool calibrating device of claim 6, wherein each said position indicating element is an infrared reflector.

10. The surgical tool calibrating device of claim 1, wherein the tool inserting portion is a calibration bore.

11. The surgical tool calibrating device of claim 1, wherein the electronic sensing module comprises a piezoelectric element.

12. The surgical tool calibrating device of claim 1, wherein the signal communication module is a wire-based communication module or a wireless communication module.
